(19) United States
(12) Patent Application Publication
(10) Pub. No.: US 2025/0195761 A1
(43) Pub. Date: Jun. 19, 2025

(54) SYSTEMS AND METHODS FOR SELECTIVELY ENABLING AND/OR DISABLING FUNCTIONALITY AND/OR SETTINGS IN A SOFTWARE UPDATE IN AN INSULIN PUMP SYSTEM

(71) Applicant: Tandem Diabetes Care, Inc., San Diego, CA (US)

(72) Inventors: Christopher JUNG, San Diego, CA (US); Russell STEPHENS, San Diego, CA (US); Bao-Tran NGUYEN, San Diego, CA (US); Justin CHOI, San Diego, CA (US); Garrett MARIN, San Diego, CA (US); Kristal Yu, San Diego, CA (US)

(73) Assignee: Tandem Diabetes Care, Inc., San Diego, CA (US)

(21) Appl. No.: 18/544,200

(22) Filed: Dec. 18, 2023

(51) Int. Cl. A61M 5/172; G16H 20/17; G16H 40/67

(52) U.S. Cl. CPC A61M 5/172; G16H 20/17; G16H 40/67

(57) ABSTRACT

Systems and methods are provided for sending a software update to an insulin pump and/or user device used with the insulin pump and selectively enabling and disabling certain functionality (e.g., operations, tasks, functions, and/or features) enabled by the software update and/or settings adjusted by the software update. The software update may be in the form of a software package that is sent from a remote device to all insulin pumps or user devices on an insulin pump network. While all devices may receive the same software update it may be desirable to restrict certain functionality and/or settings for some insulin pumps and/or user devices. The insulin pump, user device associated with the insulin pump, and/or remote device in communication with the user device, may determine whether the functionality in the software update should be disabled or otherwise not enabled for a certain insulin pump and/or user device.

### TECHNICAL FIELD

This technology generally relates to the field of insulin pump systems. For example, systems and methods are provided herein for selectively updating functionality and/or settings in devices in an insulin pump system.

### BACKGROUND

Nearly 1 in 10 individuals in the United States are affected by diabetes. As technology advances, techniques for glucose monitoring and even insulin delivery so too develop and evolve. To manage the condition, historically many of these individuals were required to administer a blood draw, for example a needle prick in the fingertip to analyze the blood to determine blood glucose levels. If the blood glucose level did not satisfy a threshold, the individual may have to administer an insulin shot, meaning an injection of insulin into the subcutaneous tissue using a needle and syringe.

With advances in insulin pump and glucose monitoring technology, individuals may opt to use a wearable insulin pump which may be in communication with a continuous glucose monitor. Wearable insulin pumps selectively deliver a bolus based on preprogrammed settings for example. Such wearable insulin pumps often introduce insulin via an infusion site and are worn on the patient's belt or sleeve or may even be adhered to the patient as a patch pump. Such wearable insulin pumps typically include suitable wireless connections for communicating with other devices and/or may communicate wirelessly with certain devices (e.g., mobile device).

Wearable insulin pumps generally include a processor, memory, and/or storage for overseeing operation of the insulin pump based on programmed parameters. Similar to other electronic devices, from time-to-time it may be necessary to update software executed on the insulin pump to adjust pump operation, include new functionality, or otherwise improve the user experience. As users of the insulin pumps have different medical needs and histories, live in various territories governed by different laws, and use their insulin pumps in different ways, it is desirable to enable certain functionality or settings on some insulin pumps but not others. However, performing software updates on only certain insulin pumps is time consuming and requires a significant amount of company resources. Further, it may be desirable to permit such restricted functionality at a later time, which further adds complexity and requires resources.

Accordingly, there is a need for systems and methods for performing a update software universally across all insulin pumps or across a large portion of insulin pumps while retaining the ability to selectively enable the updated functionality and/or settings on certain devices.

### SUMMARY

Provided herein are systems and methods for sending a software update to an insulin pump and/or user device used with the insulin pump and selectively enable and disable certain functionality (e.g., operations, tasks, functions, and/or features) enabled by the software update and/or settings adjusted or otherwise set by the software update. The software update may be in the form of a software package that is sent from a remote device to all insulin pumps and/or user devices on an insulin pump network. While all devices may receive the same software update it may be desirable to restrict certain functionality and/or settings for some insulin pumps and/or user devices. The insulin pump, user device associated with the insulin pump, and/or remote device, in communication with the user device, may determine whether the functionality in the software update should be disabled or otherwise not enabled for a certain insulin pump and/or user device. The insulin pump, user device, and/or remote device may then cause the functionality or settings to be disabled or otherwise not implemented or not enabled.

A method is provided herein for selectively enabling features of an insulin pump enabled by a software package. The method may include receiving, by an insulin pump having storage and a processor and adapted to deliver insulin to a user, the software package from a remote device or mobile device, the software package including first computer-executable instructions that when executed by the insulin pump enables a pump function, writing the computer-executable instructions to the storage to save the computer-executable instructions locally on the insulin pump, determining that the pump function is incompatible with the insulin pump, and determining not to enable the function corresponding to the first computer-executable instructions based on determining that the function is incompatible with the insulin pump, rendering the pump function inoperable on the insulin pump.

Determining that the pump function is incompatible with the insulin pump may include determining the insulin pump is incompatible with user data, pump data, medical history data, training data, and/or pump use data. The method may include determining a set time period has elapsed since determining that the pump function is incompatible with the insulin pump, and determining, after determining a set time period has elapsed, that the pump function is compatible with the insulin pump. The method may include enabling the pump function on the insulin pump, and executing the first computer-executable instructions to perform the pump function. Determining that the pump function is compatible with the insulin pump may include determining the pump function is compatible with updated user data, updated pump data, updated medical history data, updated training data, and/or updated pump use data. The method may include receiving instructions to enable the pump function on the insulin pump from the user device or the remote device, enabling the pump function on the insulin pump, and executing the first computer-executable instructions to perform the pump function. The method may include receiving instructions to enable the function on the insulin pump from an external device different from the user device and the remote device, enabling the pump function on the insulin pump, executing the first computer-executable instructions to perform the pump function.

Yet another method is provided for selectively enabling features of an insulin pump enabled by a software package. The method may include receiving, by a user device, the software package from a remote device, the software package including first computer-executable instructions that when executed by the insulin pump enables a pump function on the insulin pump, the insulin pump adapted to deliver insulin to a user, sending the software package and first instructions to insulin pump, the first instructions adapted to cause the insulin pump to save the software package locally on the insulin pump, determining that the pump function is incompatible with the insulin pump, and sending second instructions to the insulin pump to cause the insulin pump not to enable the pump function corresponding to the first computer-executable instructions based on determining that the pump function is incompatible with the insulin pump, rendering the pump function inoperable on the insulin pump.

The user device may include storage, memory, and a processor, and the method further including writing the computer-executable instructions to the storage or memory of the user device to save the computer-executable instructions locally on the user device. The method may further include determining a user record corresponding to the insulin pump, the user record including user data, pump data, medical history data, training data, and/or pump use data. Determining that the pump function is incompatible with the insulin pump may be based on the user data, pump data, medical history data, training data, and/or pump use data.

The method may further include determining a set time period has elapsed since determining that the pump function is incompatible with the insulin pump, and determining, after determining a set time period has elapsed, that the pump function is compatible with the insulin pump. Determining that the pump function is compatible with the insulin pump includes determining the pump function is compatible with updated user data, updated pump data, updated medical history data, updated training data, and/or updated pump use data associated with the user record. The method may include causing the insulin pump to enable the pump function corresponding to the first computer-executable instruction, based on determining that the pump function is compatible with the insulin pump. The method may include receiving third instructions from the remote device indicative of the insulin pump being incompatible with the pump function, wherein determining that the pump function is incompatible with the insulin pump is based on the third instructions. The method may include receiving third instructions from an external device different from the remote device, the third instructions indicative of the insulin pump being incompatible with the pump function, wherein determining that the pump function is incompatible with the insulin pump is based on the third instructions.

Yet another method is provided for selectively enabling features of an insulin pump enabled by a software package, the method including, determining, by a remote device, the software package, the software package including first computer-executable instructions that when executed by the insulin pump enables a pump function on the insulin pump, the insulin pump adapted to deliver insulin to a user, sending the software package and first instructions to a user device or insulin pump, the first instructions adapted to cause the insulin pump to save the software package locally on the insulin pump, determining that the pump function is incompatible with the insulin pump, and sending second instructions to the user device or insulin pump to cause the insulin pump to not enable the pump function corresponding to the first computer-executable instructions based on determining that the function is incompatible with the insulin pump, rendering the pump function inoperable on the insulin pump. The method further including determining a user record corresponding to the insulin pump, the user record using user data, pump data, medical history data, training data, and/or pump use data. Determining that the pump function is incompatible with the insulin pump may be based on the user data, pump data, medical history data, training data, and/or pump use data. The method may further include determining a set time period has elapsed since determining that the pump function is incompatible with the insulin pump, and determining, after determining a set time period has elapsed, that the pump function is compatible with the insulin pump based on updated user data, updated pump data, updated medical history data, updated training data, and/or updated pump use associated with the user record.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 illustrates an exemplary insulin pump system for performing a software update and determining whether certain functionality and/or settings in the software update should be enabled, in accordance with some aspects.

FIG. 2 illustrates an exemplary data flow depicting receiving a software package by an insulin pump and determining whether to enable certain functionality and/or settings in the software update.

FIG. 3 illustrates an exemplary data flow depicting receiving a software package by a user device and determining whether to enable certain functionality and/or settings in the software update.

FIG. 4 illustrates an exemplary data flow depicting determining a software package by a remote device and determining whether to enable certain functionality and/or settings in the software update.

FIG. 5 illustrates a schematic block diagram of a remote device in accordance with one or more exemplary embodiments of the disclosure.

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

The present disclosure is directed to systems and methods for providing a software update to an insulin pump and/or user device used with the insulin pump and selectively enabling and/or disabling or otherwise electing not to enable certain functionality (e.g., operations, tasks, functions, and/or features) and/or settings supported by the software update. The software update may be in the form of a software package that is sent from a remote device such as a server to all insulin pumps on an insulin pump network. For example all insulin pumps of a certain manufacturer or distributor that are the same model type may receive the software update. This may include insulin pumps in different countries or territories as well as insulin pumps used by users having different medical histories, technical proficiency, and demographics.

While all insulin pumps may receive the same software update it may be desirable to restrict certain functionality and/or settings in the software update for some insulin pumps and permit other insulin pumps to use the functionality. The remote device, user device associated with the insulin pump, or the insulin pump itself may determine whether the functionality and/or settings supported in the software update should be initially disabled or otherwise enable for a certain insulin pump. Alternatively, a device external to the insulin pump system may make this determination (e.g., a health care provider device).

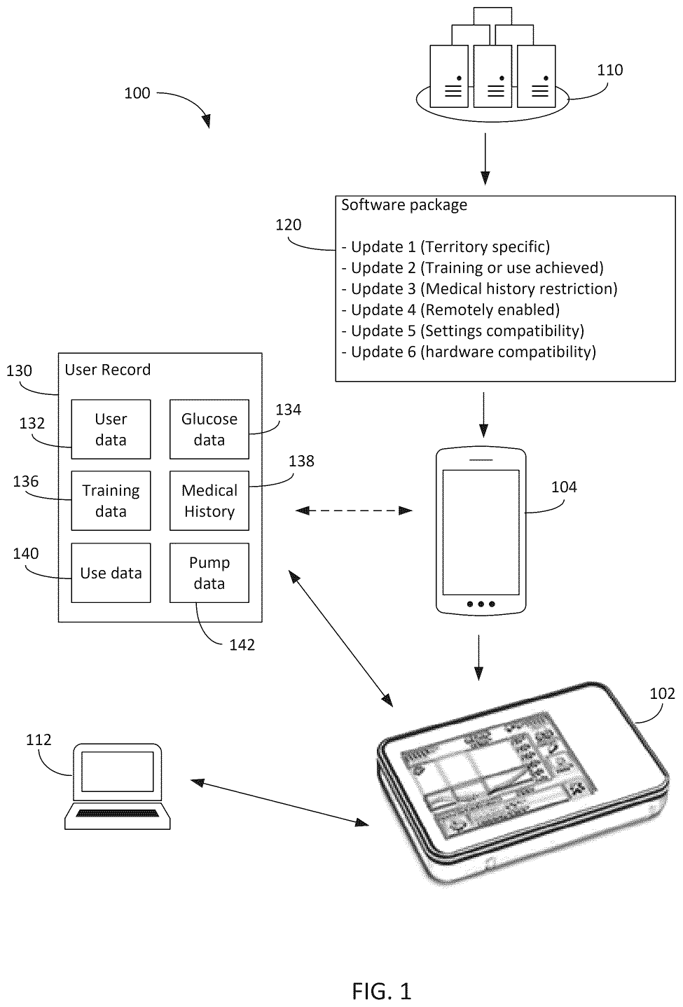

Referring now to FIG. 1, an insulin pump system for performing a software update and determining whether to enable or disable certain functionality in the software update is depicted. Insulin pump system 100 may include insulin pump 102, user device 104, and server 110. Insulin pump system 100 may also include or components of insulin pump system 100 may communicate with a glucose monitor. Components of insulin pump system 100 may communicate with external device 112, which may for example be a health care provider device. Insulin pump system 100 may include greater or fewer devices than those illustrated in FIG. 1.

Insulin pump 102 may, in one example, be the same as or similar to a t: slim X2™ insulin pump available from Tandem Diabetes Care, Inc. of San Diego, California and/or the insulin pump described in U.S. Patent App. Pub. No. 2014/0276423, published on Sep. 18, 2014 and assigned to Tandem Diabetes Care, Inc., and/or the insulin pump described in U.S. Patent App. Pub. No. 2011/014461, published on Jun. 16, 2011 and assigned to Tandem Diabetes Care, Inc., each of which are hereby incorporated by reference in their entirety. However, it is understood that insulin pump 102 may be any type of insulin pump for delivering insulin to the user and having the functionality described herein. For example, insulin pump 102 may alternatively be a patch pump that may include a pump housing connected to an adhesive patch for securing the patch pump to the user's skin. In one example, patch pump 102 may be the same as or similar to the patch pump described in U.S. Pat. No. 11,529,460 issued on Dec. 20, 2022, and assigned to AMF Medical SA, hereby incorporated by reference in its entirety.

Insulin pump 102 may include one or more processors and memory as well as a communication chip for communicating with one or more devices via a suitable wireless connection (e.g., Bluetooth, Bluetooth Low Energy (BLE), Wi-Fi Direct, any other near field communication protocol, and/or the like). For example, insulin pump 102 may communicate wirelessly with user device 104, external device 112, a continuous glucose monitor and/or other devices (e.g., other user devices). Insulin pump 102 may optionally communicate directly with remote device 110, a glucose monitoring sensor, and/or any other devices such as extremal device 112 and/or may communicate with such devices via user device 104. Insulin pump 102 may also or alternatively communicate with any device in insulin pump system 100 or any other devices via a wired connection.

User device 104 may be any suitable smart device, smart phone, mobile phone, tablet, laptop, wearable, smart device (e.g., smart watch) charging device, and/or smart sensor. User device 104 may include a computer processor, memory, and/or a communication unit which may facilitate communication with any other devices (e.g., healthcare provider device, smart sensors such as glucose monitoring sensor, and/or any other devices). User device 104 may optionally include a microphone, display (e.g., touchscreen), and/or any other input/output technology. User device 104 may communicate with any devices in insulin pump system 100 and any other devices (e.g., external device 112) via any suitable wired or wireless system (e.g., Wi-Fi, cellular network, Bluetooth, Bluetooth Low Energy (BLE), near field communication protocol, etc.).

Remote device 110 may be any computing device having a processor, memory and a communication unit. For example, remote device 110 may be one or more servers, datastores, or the like. Remote device 110 may communicate with any devices in insulin pump system 100 and any other devices (e.g., user device 104, insulin pump 102, and/or external device 112) via any well-known wired or wireless system (e.g., Wi-Fi, cellular network, Bluetooth, Bluetooth Low Energy (BLE), near field communication protocol, etc.).

Insulin pump 102 may include certain hardware and software for selectively delivering a set amount of insulin to the user based on programmed settings, which may be tailored for a given user and adjusted over time. For example, operation of insulin pump 102 may be determined and/or enabled by software and/or settings loaded onto, saved onto, and preprogrammed on insulin pump 102. Similarly, user device 104 may include certain software and/or settings for operating and/or adjusting insulin pump 102. For example, user device 104 may run and/or execute a software application that may be used to control and/or adjust insulin pump 102.

From time-to-time it may be desirable update, replace, revise, supplement, and/or supplant all or a portion of such software and/or settings on insulin pump 102 and/or user device 104. For example, software package 120 may be one or more software updates sent to user device 104 for updating the software and/or settings of user device 104 and/or insulin pump 102. Software package 120 may be generated by or otherwise originate from remote device 110 and/or sent from remote device 110 to user device 104.

As shown in FIG. 1, user device 104 may send all or a portion of software package 120 to insulin device 102. For example, remote device 110 may send instructions to user device 104 to cause user device 104 to send software package 120 insulin pump 102. Additionally or alternatively, user device 104 may send instructions to insulin pump 102 to download and/or save software package 120 locally. Alternatively, remote device 110 may send software package 120 directly to insulin pump 104 or insulin pump 102 may receive software package 120 via a wired connection.

Software package 120 may include one or more updates shown in FIG. 1. For example software package 120 may include one or more update. While six updates are shown, it is understood that more than six updates may be included. Updates may adjust the general operation of the insulin pump, the functionality of the insulin pump, insulin pump, user and/or operational settings, or may be any other adjustment or change to the operation or functionality of insulin pump 102.

Software package 120 may be sent to a large group of insulin pumps and/or user devices, though the updated functionality and/or settings may only intended to be enabled for a smaller sub-group of insulin pumps and/or users and intended to be dormant or otherwise non-functional for such other pumps and/or users. More specifically, the same software package may be sent to all insulin pumps having the same type of model, made by the same manufacturer, year model, or the like. However, one or more update in software package 120 may be restricted to users meeting certain criteria. In one example, certain functionality may be initially dormant or non-enabled for an insulin pump, but if certain criteria is later satisfied, the functionality may then be subsequently enabled.

Restrictions for a given update in software package 120 may include restrictions based on a certain territory (e.g., country, state, jurisdiction), restrictions based on whether or not the user has received certain training and/or based on a time of use or even a type of use (e.g., the way the insulin pump has been used in the past), restrictions based on medical history, medical restrictions or data, family history, and the like, restrictions based on current settings for insulin pump or even privacy or communication settings, and/or restrictions based on hardware compatibility (e.g., some insulin pumps may not support functionality in software package 120). It is understood that other restrictions and/or considerations may be used to enable and/or disable functionality (e.g., functions and/or features) and/or settings in software package 120.

To determine whether a given update in software package 120 should be enable or disabled on insulin pump 102 and/or user device 104, remote device 110, and/or insulin pump 102 may consider certain information about a user of insulin pump 104 and/or about insulin pump 102 itself. For example, user record 130 may be saved on remote device, and/or user device 104 and may contain user data 132, which may include any information about the user including a residence or location information, physiological and/or demographic information, behavioral information, health information and/or the like, glucose data 134, which may include information about the user's blood glucose levels, training data 136, which may include a record of the training received and/or provided to the user, medical history data 138, which may include the user's medical history and/or family medical history, use data 140, which may include information about how the user uses insulin pump 102 and/or the amount of user, and/or pump data 142, which may include information about operation and/or functionality of the insulin pump.

Software package 120 may include the restriction for each update or such restriction may be included in instructions provided with software package 120. Remote device 110, user device 104, and/or external device 112 may compare data in user record 130 and/or any other data relating to the user to determine whether an update meets or satisfies the restriction and if so may enable the update. Alternatively, or additionally, an update may only or optionally be enabled by external device 112 (e.g., a health care provider device).

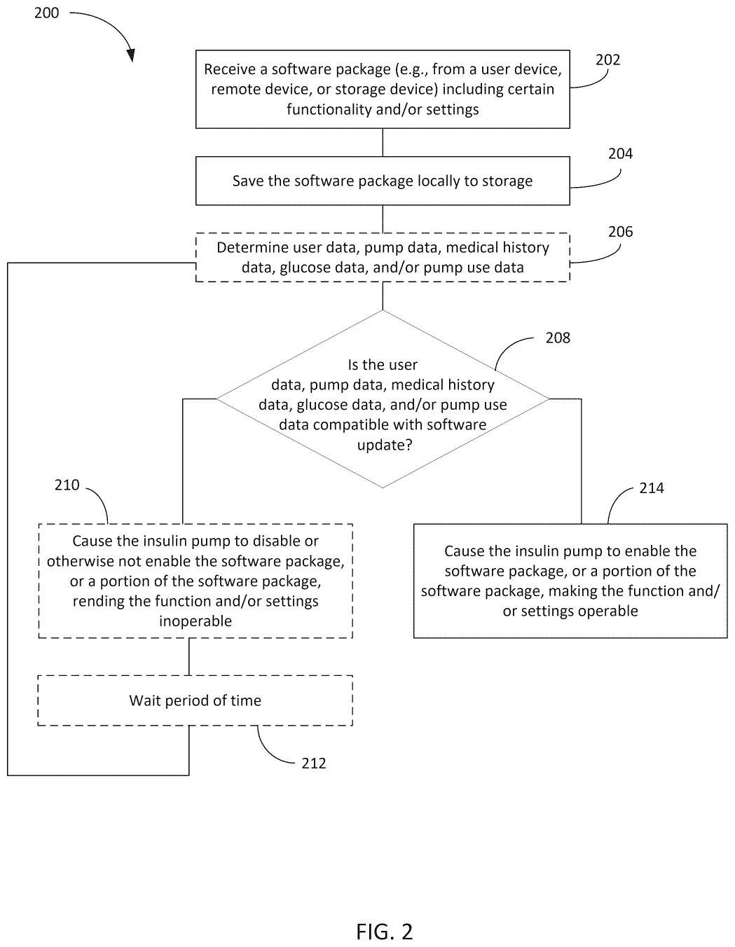

Referring now to FIG. 2, an exemplary data flow for receiving a software package by an insulin pump and determining to enable or disable certain functionality and/or settings in the software update is illustrated. Process flow 200 may be performed by an insulin pump (e.g., insulin pump 102 of FIG. 1). Alternatively, some or all of the blocks of process flow 200 in FIG. 2 may be performed in a distributed manner across any number of devices (e.g., remote device, user device, external device, etc.). Some or all of the operations of process flow 200 may be optional and may be performed in a different order.

To initiate process flow 200, at block 202, computer-executable instructions stored on a memory of a device, such as an insulin pump, may be executed to receive a software package (e.g., from a user device, a remote device and/or a storage device (e.g., USB device)), which may include certain functionality and/or settings. Instructions regarding the software package may also be received. At block 204, computer-executable instructions stored on a memory of a device, such as an insulin pump, may be executed to save the software package locally to storage and/or memory on the insulin pump. For example, the insulin pump may write the software package to storage and/or memory.

At optional block 206 computer-executable instructions stored on a memory of a device, such as an insulin pump, may be executed to determine certain user information such as user data, pump data, medical history data, glucose data, and/or pump use data. For example, a user record may be consulted and/or accessed to determine this information. At decision 208 computer-executable instructions stored on a memory of a device, such as an insulin pump, may be executed to determine whether user data, pump data, medical history data, glucose data, and/or pump use data is compatible with a software update. This decision may be made based on instructions provided to the insulin pump with the software package (e.g., from a user device or remote device) and/or may be made based on user data, pump data, medical history data, glucose data, and/or pump use data determined at optional block 206.

For example, a software package may include instructions that a certain update is incompatible with a user in a certain territory or region, a user that has not received a certain training or has not used the insulin pump for a given amount of time, a user with a certain medical history or risk label, a user that has certain user or pump settings, or a user of a certain insulin pump model and/or hardware.

If the user data, pump data, medical history data, glucose data, and/or pump use data is not compatible with the software update, then at optional block 210 computer-executable instructions stored on a memory of a device, such as an insulin pump, may be executed to cause the insulin pump to disable or otherwise cause the insulin pump not to enable the software or a portion of the software package (e.g., the incompatible update), thereby rendering the functionality and/or settings in the update in operable. At optional block 212 computer-executable instructions stored on a memory of a device, such as an insulin pump, may be executed to cause the insulin pump to wait a set period of time (one day, one week, one month, etc.) and then reinitiate block 206.

Alternatively, if the user data, pump data, medical history data, glucose data, and/or pump use data is compatible with the software update, then at option block 214 computer-executable instructions stored on a memory of a device, such as an insulin pump, may be executed to cause the insulin pump to enable the software package, or a portion of the software package, making the function and/or settings operable.

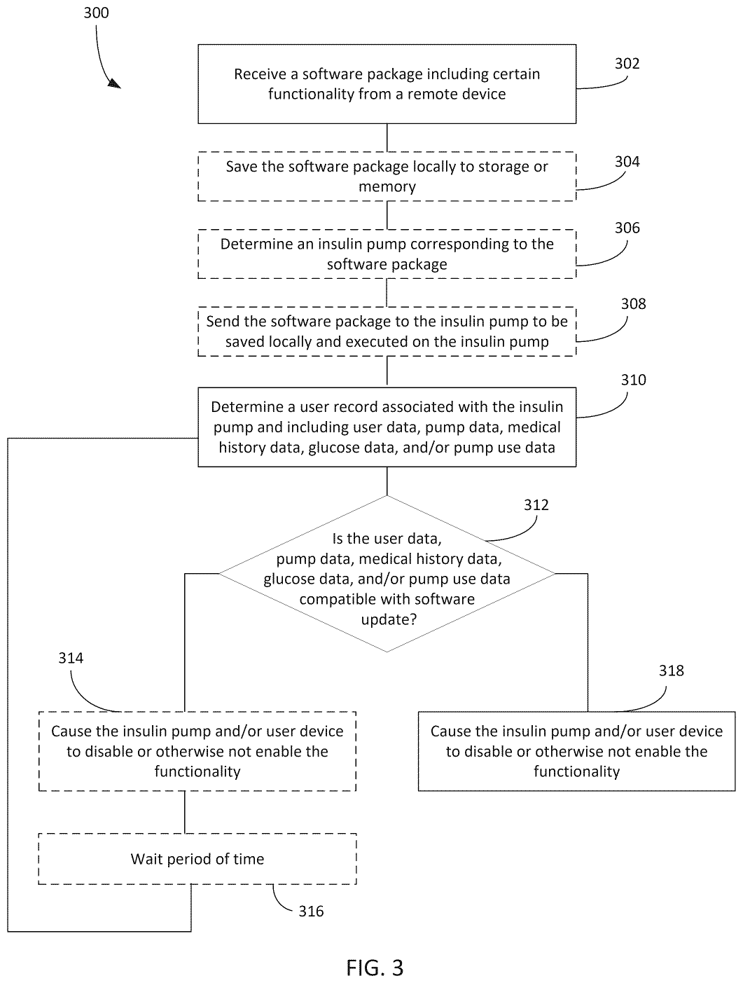

Referring now to FIG. 3, an exemplary data flow depicting receiving a software package by a user device and determining to enable or disable certain functionality in the software update is illustrated. Process flow 300 may be performed by a user device (e.g., user device 104 of FIG. 1). Alternatively, some or all of the blocks of process flow 300 in FIG. 3 may be performed in a distributed manner across any number of devices (e.g., remote device, insulin pump, external device, etc.). Some or all of the operations of process flow 200 may be optional and may be performed in a different order.

To initiate process flow 300, at block 302, computer-executable instructions stored on a memory of a device, such as a user device, may be executed to receive a software package (e.g., software update) from a remote device. The software package may include updates for the user device and/or insulin pump for updating certain functionality or settings. At optional block 304, computer-executable instructions stored on a memory of a device, such as a user device, may be executed to save the software package locally on the user device if all or a portion of the software package is intended for user device 104.

At optional block 306, computer-executable instructions stored on a memory of a device, such as a user device, may be executed to determine an insulin pump corresponding to the software package. For example, an insulin pump associated with the user device (e.g., in a user profile or otherwise) may be determined. At optional block 308, computer-executable instructions stored on a memory of a device, such as a user device, may be executed to send the software package or a portion thereof to the insulin pump to be save locally on the insulin pump. Instructions to cause the insulin pump to save the software package locally may also be sent to the insulin pump.

At decision 310, computer-executable instructions stored on a memory of a device, such as a user device, may be executed to determine that a user record associated with the insulin pump includes user data, pump data, medical history data, glucose data, and/or pump use data. The user record may be saved on the user device and/or a remote device and accessed by the user device. At decision 312, computer-executable instructions stored on a memory of a device, such as a user device, may be executed to determine whether the user data, pump data, medical history data, glucose data, and/or pump use data are compatible with the software update. This decision may be made based on instructions provided to the insulin pump with the software package (e.g., from the remote device) and/or may be made based on user data, pump data, medical history data, glucose data, and/or pump use data determined at optional block 310.

If the user data, pump data, medical history data, glucose data, and/or pump use data is not compatible with the software update, then at optional block 314 computer-executable instructions stored on a memory of a device, such as a user device, may be executed to cause the user device and/or insulin pump to disable the software or otherwise cause the software or a portion of the software package (e.g., the incompatible update) to not be enabled, thereby rendering the functionality and/or settings in the update inoperable. For example, the user device may send instructions to the insulin pump to cause insulin pump not to enable the update in the software package. At optional block 316 computer-executable instructions stored on a memory of a device, such as a user device, may be executed to cause the insulin pump to wait a set period of time (one day, one week, one month, etc.) and then reinitiate block 310.

Alternatively, if the user data, pump data, medical history data, glucose data, and/or pump use data is compatible with the software update or a portion thereof, then at option block 318 computer-executable instructions stored on a memory of a device, such as a user device, may be executed to cause the insulin pump and user device to enable the software package, or a portion of the software package, making the compatible function and/or settings operable. For example, the user device may send instructions to the insulin pump to enable the update in the software package.

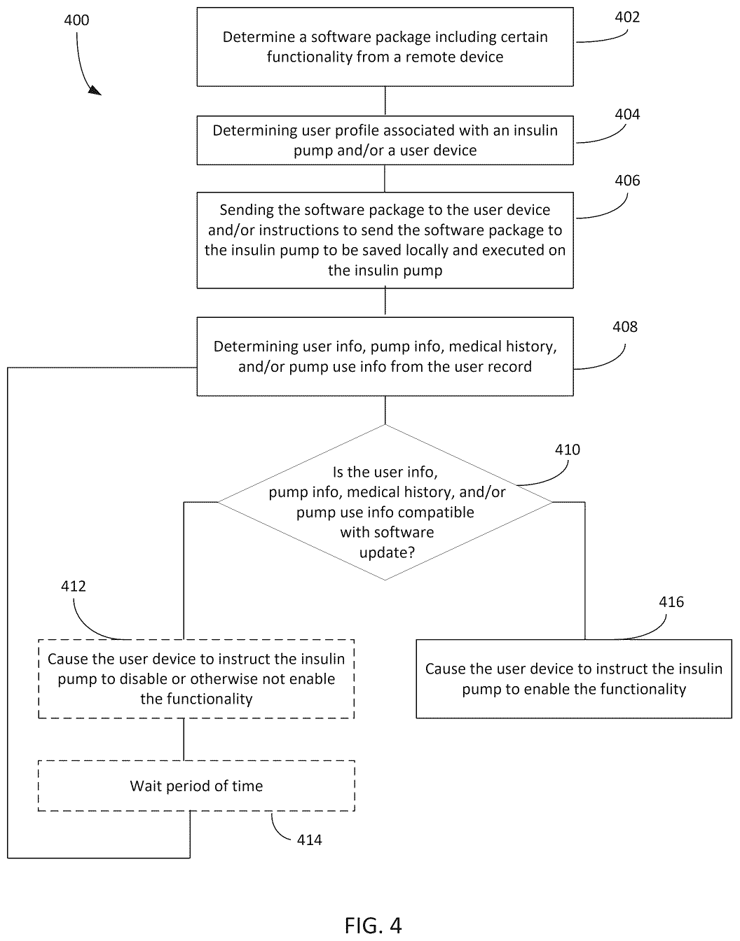

Referring now to FIG. 4, an exemplary data flow for determining a software package by a remote device and determining to enable or disable certain functionality and/or settings in the software update is illustrated. Process flow 400 may be performed by a remote device (e.g., remote device 110 of FIG. 1). Alternatively, some or all of the blocks of process flow 400 in FIG. 4 may be performed in a distributed manner across any number of devices (e.g., insulin pump, user device, external device, etc.). Some or all of the operations of process flow 400 may be optional and may be performed in a different order.

To initiate process flow 400, at block 402, computer-executable instructions stored on a memory of a device, such as a remote device, may be executed to determine a software package (e.g., software update). The software package may include updates for the user device and/or insulin pump for updating certain functionality or settings. At optional block 404, computer-executable instructions stored on a memory of a device, such as a remote device, may be executed to determine a user profile associated with an insulin pump and/or a user device.

At block 406, computer-executable instructions stored on a memory of a device, such as a remote device, may be executed to send the software package or a portion thereof to the user device with instructions to save the software package locally on the user device and/or to send the software package to the insulin pump. Instructions to cause the insulin pump to save the software package locally may also be sent to the user device.

At block 408, computer-executable instructions stored on a memory of a device, such as a remote device, may be executed to determine a user record associated with the insulin pump and including user data, pump data, medical history data, glucose data, and/or pump use data. The user record may be saved on the remote device and/or accessed by the remote device. At decision 410, computer-executable instructions stored on a memory of a device, such as a remote device, may be executed to determine whether the user data, pump data, medical history data, glucose data, and/or pump use data is compatible with the software update. This decision may be made based on the user data, pump data, medical history data, glucose data, and/or pump use data determined at optional block 408.

If the user data, pump data, medical history data, glucose data, and/or pump use data is not compatible with the software update, then at optional block 412 computer-executable instructions stored on a memory of a device, such as a remote device, may be executed to cause the user device and/or insulin pump to disable the software or otherwise cause the software or a portion of the software package (e.g., the incompatible update) not to be enabled, thereby rendering the functionality and/or settings in the update inoperable. For example, the remote device may send instructions to the user device and/or insulin pump to disable one or more update in the software package or otherwise cause one or more update in the software package not to be enabled. At optional block 414 computer-executable instructions stored on a memory of a device, such as a remote device, may be executed to cause the insulin pump to wait a set period of time (one day, one week, one month, etc.) and then reinitiate block 408.

Alternatively, if the user data, pump data, medical history data, glucose data, and/or pump use data is compatible with the software update or a portion thereof, then at option block 416 computer-executable instructions stored on a memory of a device, such as a remote device, may be executed to cause the user device and/or insulin pump to enable the software package, or a portion of the software package, making the compatible function and/or settings operable. For example, the remote device may send instructions to the user device and/or the insulin pump (e.g., via the user device) to enable the update in the software package.

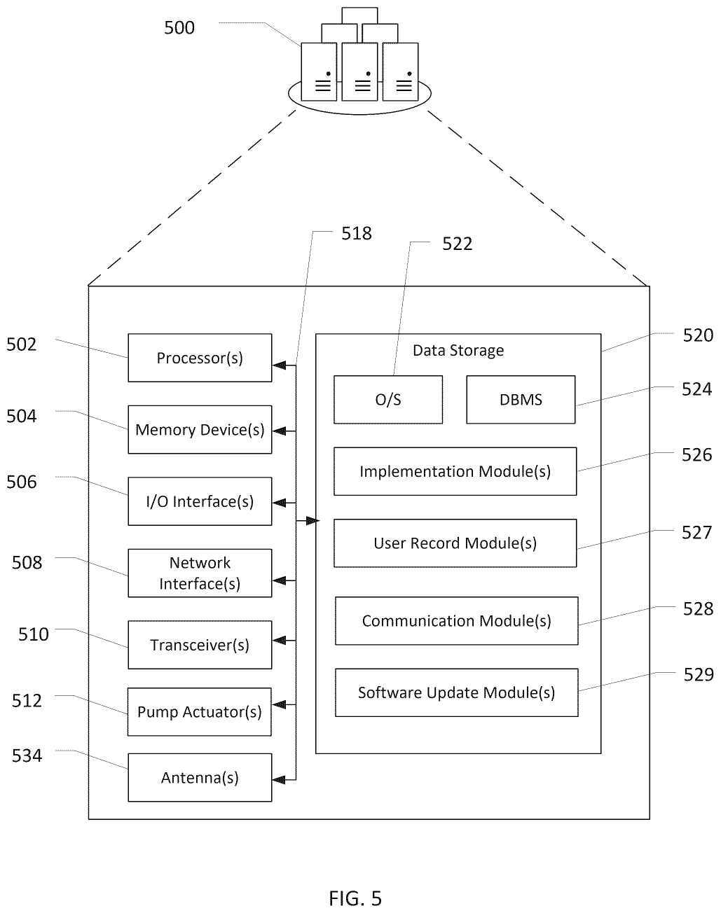

FIG. 5 is a schematic block diagram of illustrative remote device 500, which may be in communication with one or more device in insulin pump system (e.g., user device, insulin pump device, etc.), is illustrated. Remote device 500 may be the same or similar to remote device 110 of FIG. 1 or otherwise one or more of the remote devices of FIGS. 2-4. It is understood that remote device 500 may alone or together with one or more device of the insulin pump system perform one or more of the operations of remote device 500. For example, a user device (e.g., user device 104 of FIG. 1) and/or insulin pump (e.g., insulin pump 102 of FIG. 1) may perform one or more of the operations and tasks of the remote device.

Remote device 500 may be designed to communicate with one or more periphery devices, remote devices, smart devices, computing devices, servers, other systems, or the like. Remote device 500 may be designed to communicate via one or more networks. Such network(s) may include, but are not limited to, any one or more different types of communications networks such as, for example, near field communication networks, cable networks, public networks (e.g., the Internet), private networks (e.g., frame-relay networks), wireless networks, cellular networks, telephone networks (e.g., a public switched telephone network), or any other suitable private or public packet-switched or circuit-switched networks.

In an illustrative configuration, remote device 500 may include one or more processors 502, one or more memory devices 506 (also referred to herein as memory 506), one or more input/output (I/O) interface(s) 506, one or more network interface(s) 508, one or more transceiver(s) 510, one or more pump actuator(s) 512, one or more antenna(s) 534, and data storage 520. Remote device 500 may further include one or more bus(es) 518 that functionally couple various components of the remote device 500.

The bus(es) 518 may include at least one of a system bus, a memory bus, an address bus, or a message bus, and may permit exchange of information (e.g., data (including computer-executable code), signaling, etc.) between various components of the remote device 500. The bus(es) 518 may include, without limitation, a memory bus or a memory controller, a peripheral bus, an accelerated graphics port, and so forth. The bus(es) 518 may be associated with any suitable bus architecture including.

The memory 506 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In various implementations, the memory 506 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth.

The data storage 520 may include removable storage and/or non-removable storage including, but not limited to, magnetic storage, optical disk storage, and/or tape storage. The data storage 520 may provide non-volatile storage of computer-executable instructions and other data. The memory 506 and the data storage 520, removable and/or non-removable, are examples of computer-readable storage media (CRSM) as that term is used herein. The data storage 520 may store computer-executable code, instructions, or the like that may be loadable into the memory 506 and executable by the processor(s) 502 to cause the processor(s) 502 to perform or initiate various operations. The data storage 520 may additionally store data that may be copied to memory 506 for use by the processor(s) 502 during the execution of the computer-executable instructions. Moreover, output data generated as a result of execution of the computer-executable instructions by the processor(s) 502 may be stored initially in memory 506, and may ultimately be copied to data storage 520 for non-volatile storage.

The data storage 520 may store one or more operating systems (O/S) 522; one or more optional database management systems (DBMS) 524; and one or more program module(s), applications, engines, computer-executable code, scripts, or the like such as, for example, one or more implementation modules 526, one or more user record module 527, one or more communication modules 528, and/or one or more software update modules 529. Some or all of these modules may be sub-modules. Any of the components depicted as being stored in data storage 520 may include any combination of software, firmware, and/or hardware. The software and/or firmware may include computer-executable code, instructions, or the like that may be loaded into the memory 506 for execution by one or more of the processor(s) 502. Any of the components depicted as being stored in data storage 520 may support functionality described in reference to correspondingly named components earlier in this disclosure.

Referring now to other illustrative components depicted as being stored in data storage 520, O/S 522 may be loaded from data storage 520 into memory 506 and may provide an interface between other application software executing on remote device 500 and hardware resources of the remote device 500. More specifically, O/S 522 may include a set of computer-executable instructions for managing hardware resources of remote device 500 and for providing common services to other application programs (e.g., managing memory allocation among various application programs). In certain example embodiments, O/S 522 may control execution of the other program module(s) for content rendering. O/S 522 may include any operating system now known or which may be developed in the future including, but not limited to, any server operating system, any mainframe operating system, or any other proprietary or non-proprietary operating system.

Optional DBMS 524 may be loaded into the memory 506 and may support functionality for accessing, retrieving, storing, and/or manipulating data stored in memory 506 and/or data stored in data storage 520. DBMS 524 may use any of a variety of database models (e.g., relational model, object model, etc.) and may support any of a variety of query languages. DBMS 524 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like.

I/O interface(s) 506 may facilitate the receipt of input information by remote device 500 from one or more I/O devices as well as the output of information from remote device 500 to the one or more I/O devices. The I/O devices may include any of a variety of components such as a touchscreen display; an audio output device for producing sound, such as a speaker; an audio capture device, such as a microphone; buttons and/or dials; and so forth. Any of these components may be integrated into remote device 500 or may be separate.

Remote device 500 may further include one or more network interface(s) 508 via which remote device 500 may communicate with any of a variety of other systems, platforms, networks, devices, and so forth. Network interface(s) 508 may enable communication, for example, with one or more wireless routers, one or more host servers, one or more web servers, and the like via one or more of networks.

Antenna(s) 534 may include any suitable type of antenna depending, for example, on the communications protocols used to transmit or receive signals via antenna(s) 534. Non-limiting examples of suitable antennas may include directional antennas, non-directional antennas, dipole antennas, folded dipole antennas, patch antennas, multiple-input multiple-output (MIMO) antennas, or the like. Antenna(s) 534 may be communicatively coupled to one or more transceivers 510 or radio components to which or from which signals may be transmitted or received. Antenna(s) 534 may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals including BLE signals, a Near Field Communication (NFC) antenna for transmitting or receiving NFC signals, a 900 MHz antenna, and so forth.

Transceiver(s) 510 may include any suitable radio component(s) for, in cooperation with the antenna(s) 534, transmitting or receiving radio frequency (RF) signals in the bandwidth and/or channels corresponding to the communications protocols utilized by remote device 500 to communicate with other devices. Transceiver(s) 510 may include hardware, software, and/or firmware for modulating, transmitting, or receiving—potentially in cooperation with any of antenna(s) 534—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. Transceiver(s) 510 may further include hardware, firmware, or software for receiving GNSS signals. Transceiver(s) 510 may include any known receiver and baseband suitable for communicating via the communications protocols utilized by the remote device 500. The transceiver(s) 510 may further include a low noise amplifier (LNA), additional signal amplifiers, an analog-to-digital (A/D) converter, one or more buffers, a digital baseband, or the like.

Referring now to functionality supported by the various program module(s) depicted in FIG. 6, implementation module(s) 526 may include computer-executable instructions, code, or the like that responsive to execution by one or more of the processor(s) 502 may perform functions including, but not limited to, overseeing coordination and interaction between one or more modules and computer executable instructions in data storage 520, determining user actions, determining actions associated with user interactions, determining actions associated with user input, initiating commands locally or at periphery and/or remote devices, and the like.

The user record module(s) 527 may include computer-executable instructions, code, or the like that responsive to execution by one or more of the processor(s) 502 may perform functions including, but not limited to maintaining user records for each user in the insulin pump system, platform, and/or network which may include user data, glucose data, training data, medical history data, use data, and/or pump data.

Communication module(s) 528 may include computer-executable instructions, code, or the like that responsive to execution by one or more of the processor(s) 502 may perform functions including, but not limited to, communicating with one or more devices, for example, via wired or wireless communication, communicating with user devices (e.g., mobile devices), smart devices, remote devices, charging devices, computing devices, smart sensors and/or any other devices.

Software update module(s) 529 may include computer-executable instructions, code, or the like that responsive to execution by one or more of the processor(s) 502 may perform functions including, but not limited to, maintaining software records for each device in the insulin pump system and cause devices in the insulin pump system such as user devices and insulin pumps to perform software updates (e.g., using software packages) to update the functionality and/or settings and the like on such devices.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Program module(s), applications, or the like disclosed herein may include one or more software components, including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component including assembly language instructions may require conversion into executable machine code by an assembler prior to execution by the hardware architecture and/or platform.

Another example programming language may be a higher-level programming language that may be portable across multiple architectures. A software component including higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component including instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may include other custom-developed application software, operating system functionality (e.g., device drivers, data storage (e.g., file management) routines, other common routines, and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a CRSM that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method for selectively enabling features of an insulin pump enabled by a software package, the method comprising:
receiving, by an insulin pump having storage and a processor and adapted to deliver insulin to a user, the software package from a remote device or mobile device, the software package comprising first computer-executable instructions that when executed by the insulin pump enables a pump function;
writing the computer-executable instructions to the storage to save the computer-executable instructions locally on the insulin pump;
determining that the pump function is incompatible with the insulin pump; and
determining not to enable the pump function corresponding to the first computer-executable instructions based on determining that the pump function is incompatible with the insulin pump, rendering the pump function inoperable on the insulin pump.

2. The method of claim 1, wherein determining that the pump function is incompatible with the insulin pump comprises determining the insulin pump is incompatible with user data, pump data, medical history data, training data, and/or pump use data.

3. The method of claim 2, further comprising:
determining set time period has elapsed since determining that the pump function is incompatible with the insulin pump; and
determining, after determining a set time period has elapsed, that the pump function is compatible with the insulin pump.

4. The method of claim 2, further comprising:
enabling the pump function on the insulin pump; and
executing the first computer-executable instructions to perform the pump function.

5. The method of claim 3, wherein determining that the pump function is compatible with the insulin pump comprises determining the pump function is compatible with updated user data, updated pump data, updated medical history data, updated training data, and/or updated pump use data.

6. The method of claim 1, further comprising:
receiving instructions to enable the pump function on the insulin pump from the user device or the remote device;
enabling the pump function on the insulin pump; and
executing the first computer-executable instructions to perform the pump function.

7. The method of claim 1, further comprising:
receiving instructions to enable the pump function on the insulin pump from an external device different from the user device and the remote device;
enabling the pump function on the insulin pump; and
executing the first computer-executable instructions to perform the pump function.

8. A method for selectively enabling features of an insulin pump enabled by a software package, the method comprising:
receiving, by a user device, the software package from a remote device, the software package comprising first computer-executable instructions that when executed by the insulin pump enables a pump function on the insulin pump, the insulin pump adapted to deliver insulin to a user;
sending the software package and first instructions to insulin pump, the first instructions adapted to cause the insulin pump to save the software package locally on the insulin pump;
determining that the pump function is incompatible with the insulin pump; and
sending second instructions to the insulin pump to cause the insulin pump not to enable the pump function corresponding to the first computer-executable instructions based on determining that the pump function is incompatible with the insulin pump, rendering the pump function inoperable on the insulin pump.

9. The method of claim 8, wherein the user device comprises storage, memory, and a processor, and the method further comprising writing the computer-executable instructions to the storage or memory of the user device to save the computer-executable instructions locally on the user device.

10. The method of claim 8, further comprising determining a user record corresponding to the insulin pump, the user record comprising user data, pump data, medical history data, training data, and/or pump use data.

11. The method of claim 10, wherein determining that the pump function is incompatible with the insulin pump is based on the user data, pump data, medical history data, training data, and/or pump use data.

12. The method of claim 10, further comprising:
determining a set time period has elapsed since determining that the pump function is incompatible with the insulin pump; and
determining, after determining a set time period has elapsed, that the pump function is compatible with the insulin pump.

13. The method of claim 12, wherein determining that the pump function is compatible with the insulin pump comprises determining the pump function is compatible with updated user data, updated pump data, updated medical history data, updated training data, and/or updated pump use data associated with the user record.

14. The method of claim 10, further comprising causing the insulin pump to enable the pump function corresponding to the first computer-executable instruction, based on determining that the pump function is compatible with the insulin pump.

15. The method of claim 8, further comprising receiving third instructions from the remote device indicative of the insulin pump being incompatible with the pump function, wherein determining that the pump function is incompatible with the insulin pump is based on the third instructions.

16. The method of claim 8, further comprising receiving third instructions from an external device different from the remote device, the third instructions indicative of the insulin pump being incompatible with the pump function, wherein determining that the pump function is incompatible with the insulin pump is based on the third instructions.

17. A method for selectively enabling features of an insulin pump enabled by a software package, the method comprising:
determining, by a remote device, the software package, the software package comprising first computer-executable instructions that when executed by the insulin pump enables a pump function on the insulin pump, the insulin pump adapted to deliver insulin to a user;
sending the software package and first instructions to a user device or insulin pump, the first instructions adapted to cause the insulin pump to save the software package locally on the insulin pump;
determining that the pump function is incompatible with the insulin pump; and
sending second instructions to the user device or insulin pump to cause the insulin pump not to enable the pump function corresponding to the first computer-executable instructions based on determining that the pump function is incompatible with the insulin pump, rendering the pump function inoperable on the insulin pump.

18. The method of claim 17, further comprising determining a user record corresponding to the insulin pump, the user record comprising user data, pump data, medical history data, training data, and/or pump use data.

19. The method of claim 18, wherein determining that the pump function is incompatible with the insulin pump is based on the user data, pump data, medical history data, training data, and/or pump use data.

20. The method of claim 19, further comprising:
determining a set time period has elapsed since determining that the pump function is incompatible with the insulin pump; and
determining, after determining a set time period has elapsed, that the pump function is compatible with the insulin pump based on updated user data, updated pump data, updated medical history data, updated training data, and/or updated pump use associated with the user record.
